(19) United States
(12) Patent Application Publication
(10) Pub. No.: US 2004/0023983 A1
(43) Pub. Date: Feb. 5, 2004

(54) Methods of use of fluoroquinolone compounds against pathogenic helicobacter bacteria

(76) Inventors: Alison F. Chalker, Collegeville, PA (US); Heather W. Minehart, Collegeville, PA (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/416,179

(22) PCT Filed: Nov. 8, 2001
(86) PCT No.: PCT/US01/47460

(51) Int. Cl. A61K 31/496

(52) U.S. Cl. 514/253.08

(57) ABSTRACT

This invention relates, in part, to newly identified methods of using quinolone antibiotics, particularly a gemifloxacin compound against certain pathogenic bacteria.

This invention relates, in part, to newly identified methods of using quinolone antibiotics, particularly a gemifloxacin compound against Helicobacter bacteria, such as Helicobacter sp., H. pylori, and pathogenic species of Helicobacter.

### BACKGROUND OF THE INVENTION

Quinolones have been shown to be effective to varying degrees against a range of bacterial pathogens. However, as diseases caused by these pathogens are on the rise, there exists a need for antimicrobial compounds that are more potent than the present group of quinolones.

Gemifloxacin mesylate is a novel fluoroquinolone useful as a potent antibacterial agent. Gemifloxacin compounds are described in detail in patent application PCT/KR98/00051 published as WO 98/42705. Patent application EP 688772 discloses novel quinoline(naphthyridine)carboxylic acid derivatives, including anhydrous (R,S)-7-(3-aminomethyl-4-methoxyiminopyrrolidin-1-yl)-1-cyclopropyl-6-fluoro-4-oxo-1,4-dihydro-1,8-naphthyridine-3-carboxylic acid of formula I.

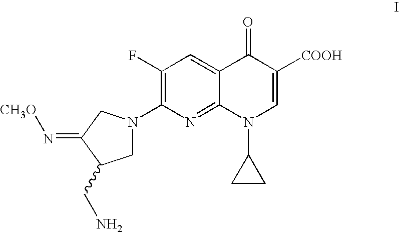

PCT/KR98/00051 discloses (R,S)-7-(3-aminomethyl-4-syn-methoxyimino-pyrrolidin-1-yl)-1-cyclopropyl-6-fluoro-4-oxo-1,4-dihydro-1,8-naphthyridine-3-carboxylic acid methanesulfonate and hydrates thereof including the sesquihydrate. Provided herein is a significant discovery made using a gemifloxacin compound against Helicobacter, demonstrating the activity of the gemifloxacin compound used was superior to a number of quinolones as described in more detail herein. Gemifloxacin compounds are valuable compounds for the treatment of bacterial infection caused by a range of Helicobacter pathogens, including those resistant to usual oral therapy, thereby filling an unmet medical need.

### SUMMARY OF THE INVENTION

An object of the invention is a method for modulating metabolism of pathogenic Helicobacter bacteria comprising the step of contacting pathogenic Helicobacter bacteria with an antibacterially effective amount of a composition comprising a quinolone, particularly a gemifloxacin compound, or an antibacterially effective derivative thereof.

A further object of the invention is a method wherein said pathogenic Helicobacter bacteria is selected from the group consisting of: members of the genus Helicobacter, Helicobacter sp, H. pylori, and a pathogenic species of Helicobacter. Also provided by the invention is a method of treating or preventing a bacterial infection by pathogenic Helicobacter bacteria comprising the step of administering an antibacterially effective amount of a composition comprising a quinolone, particularly a gemifloxacin compound to a mammal suspected of having or being at risk of having an infection with pathogenic Helicobacter bacteria.

A preferred method is provided wherein said modulating metabolism is inhibiting growth of said bacteria or killing said bacteria.

A further preferred method is provided wherein said contacting said bacteria comprises the further step of introducing said composition into a mammal, particularly a human.

Further preferred methods are provided by the invention wherein said bacteria is selected from the group consisting of: members of the genus Helicobacter, Helicobacter sp, H. pylori, and a pathogenic species of Helicobacter.

For administration, the preferred composition of the invention include, among other things, methods wherein said composition comprises gemifloxacin, or a pharmaceutically acceptable derivative thereof. The invention also provides methods wherein the gemifloxacin compound is gemifloxacin or a pharmaceutically acceptable salt thereof, or a gemifloxacin compound such as gemifloxacin mesylate or a hydrate thereof, and/or the gemifloxacin compound is gemifloxacin mesylate sesquihydrate.

Various changes and modifications within the spirit and scope of the disclosed invention will become readily apparent to those skilled in the art from reading the following descriptions and from reading the other parts of the present disclosure.

### DESCRIPTION OF THE INVENTION

The present invention provides, among other things, methods for using a composition comprising a quinolone, particularly a gemifloxacin compound against a range of pathogenic bacteria.

As used herein “gemifloxacin compound(s)” means a compound having antibacterial activity described in patent application PCT/KR98/00051 published as WO 98/42705, or patent application EP 688772.

This invention was based, in part, on analyses evaluating the in vitro activity of a gemifloxacin compound, as well as other new quinolones using low-passaged clinical isolates and type strains of Helicobacter species commonly found in the gastrointestinal tract of humans. Organisms used in the analyses included: members of the genus Helicobacter, Helicobacter sp, H. pylori, and a pathogenic species of Helicobacter. The common gastric pathogen Helicobacter pylori is implicated in the development of chronic gastritis, duodenal and gastric ulcers and gastric mucosa-associated lymphoid tissue (MALT) lymphoma, and there is an established link between H. pylori infection and gastric cancer. Eradication of the infection is often recommended for symptomatic H. pylori-positive patients, particularly those with duodenal or gastric ulcer. However, the efficacy of common triple therapy treatments for H. pylori is being undermined by the rise in frequency of antibiotic resistant isolates. Although resistance to amoxicillin and tetracycline is rare, substantial and increasing rates of resistance to metronidazole and clarithromycin are a significant cause of treatment failure (Alarcon, T., Domingo, D., & Lopez-Brea, M. 1999. International Journal of Antimicrobial Agents 12, 19-26). Hence there is considerable interest in identifying alternative therapies which are more effective against H. pylori.

Fluoroquinolones are widely used for treatment of gastrointestinal, respiratory and urinary tract infections. Although they are not commonly used to treat H. pylori infections, fluoroquinolones are active in vitro against H. pylori. Recently, the susceptibility of 57 strains of H. pylori taken from human gastric biopsies to 11 antimicrobial agents including 4 fluoroquinolones was determined (Sanchez, J. E., Saenz, N. G., Rincon, M. R., Martin, I. T., Sanchez, E. G., & Martinez, M. J. 2000. Journal of Antimicrobial Chemotherapy 46, 283-5).

The aim of the present invention is to evaluate the susceptibility of H. pylori to the new fluoroquinolone gemifloxacin (SB-265805). Twenty-one (21) H. pylori strains were obtained, including 16 clinical isolates CCUG 39297, CCUG 30988 (Lelwala-Guruge, J., Ljungh, A., & Wadstrom, T. 1992. APMIS 100, 908-13), CCUG 17875 (Lelwala-Guruge, J., Ljungh, A., & Wadstrom, T. 1992. APMIS 100, 908-13; Ilver, D., Arnqvist, A., Ogren, J., Frick, I. M., Kersulyte, D., Incecik, E. T. et al. 1998. Science 279, 373-7), CCUG 38771, CCUG 15818, CCUG 17874 (Lelwala-Guruge, J., Ljungh, A., & Wadstrom, T. 1992. APMIS 100, 908-13; O'Toole, P. W., Logan, S. M., Kostrzynska, M., Wadström, T., & Trust, T. J. 1991. Journal of Bacteriology 173, 505-513), CCUG 19087, CCUG 30987 (Lelwala-Guruge, J., Ljungh, A., & Wadstrom, T. 1992. APMIS 100, 908-13), CCUG 38772, CCUG 18943, CCUG 18295, CCUG 36718, CCUG 36719, CCUG 35899, CCUG 36720, CCUG 39924; and laboratory strains Not18, Not34, ATCC 43504, SS1, and ATCC 700392/26695. These strains include gastric biopsy isolates from the USA, Sweden, France, Belgium, UK, South Africa, and Australia Two of the clinical isolates are reported to have the cagA⁺ genotype (strains CCUG 17874 and CCUG 17875) and three to be cagA⁻ (strains CCUG 36720, CCUG 36719, and CCUG 30988), but the cag genotype of the rest is unknown. The NCCLS recommended agar dilution method (National Committee for Clinical Laboratory Standards. 2000. Approved Standard—Fifth edition. M7-A5 and MIC Testing Supplemental Tables M100-S10 (M7) Vol. 20(2). NCCLS, Villanova, Pa.) was used to determine the activities of gemifloxacin and comparator drugs also useful in the methods of the invention. These comparator drugs, to which gemifloxacin was compared, include levofloxacin, ciprofloxacin, moxifloxacin and gatifloxacin. The activities of amoxycillin, tetracycline and metronidazole against H. pylori ATCC 43504 were also tested as controls. The agar plates were inoculated using a Steers replicator and read at 72 hours.

The control antibiotics amoxycillin, tetracycline and metronidazole gave MICs against H. pylori ATCC 43504 of 0.06, 1.0 and 256 mg/L respectively, which are within NCCLS quality control breakpoint limits. Gemifloxacin was the most active of the fluoroquinolones tested, followed by gatifloxacin, ciprofloxacin, levofloxacin and moxifloxacin; and was highly active against all H. pylori strains tested, with a MIC₉₀ of 0.13 mg/L (Table 1).

TABLE 1
MICs (mg/L) of 21 isolates of H. pylori to fluoroquinolones.

| | Range | MIC₅₀ | MIC₉₀ |
|---|---|---|---|
| Gemifloxacin | 0.03-1.0 | 0.06 | 0.13 |
| Levofloxacin | 0.13-2.0 | 0.25 | 0.5 |
| Ciprofloxacin | 0.06->2.0 | 0.25 | 0.5 |
| Moxifloxacin | 0.13-2.0 | 0.25 | 0.5 |
| Gatifloxacin | 0.03-0.5 | 0.13 | 0.13 |

There was no apparent correlation of MIC with cag status or geographical location. In conclusion it was found that, in comparison with commonly prescribed fluoroquinolones, gemifloxacin demonstrated greater activity against H. pylori strains including gastric biopsy isolates from various locations worldwide.

The invention provides a method for modulating metabolism of pathogenic Helicobacter bacteria. Skilled artisans can readily choose pathogenic Helicobacter bacteria or patients infected with or suspected to be infected with these organisms to practice the methods of the invention. Alternatively, the bacteria useful in the methods of the invention may be those described herein.

The contacting step in any of the methods of the invention may be performed in many ways that will be readily apparent to the skilled artisan. However, it is preferred that the contacting step is a provision of a composition comprising a gemifloxacin compound to a human patient in need of such composition or directly to bacteria in culture medium or buffer.

For example, when contacting a human patient or contacting said bacteria in a human patient or in vitro, the compositions comprising a quinolone, particularly a gemifloxacin compound, preferably pharmaceutical compositions may be administered in any effective, convenient manner including, for instance, administration by topical, oral, anal, vaginal, intravenous, intraperitoneal, intramuscular, subcutaneous, intranasal or intradermal routes among others.

It is also preferred that these compositions be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a compound of the invention, a quinolone, preferably a gemifloxacin compound, and a pharmaceutically acceptable carrier or excipient. Such carriers may include, but are not limited to, saline, buffered saline, dextrose, water, glycerol, ethanol and combinations thereof. The formulation should suit the mode of administration.

Quinolone compounds, particularly gemifloxacin compounds and compostions of the methods of the invention may be employed alone or in conjunction with other compounds, such as bacterial efflux pump inhibitor compounds or antibiotic compounds, particularly non-quinolone compounds, e.g., beta-lactam antibiotic compounds.

In therapy or as a prophylactic, the active agent of a method of the invention is preferably administered to an individual as an injectable composition, for example as a sterile aqueous dispersion, preferably an isotonic one.

Alternatively, the gemifloxacin compounds or compositions in the methods of the invention may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to mammals, and particularly humans, it is expected that the antibacterially effective amount is a daily dosage level of the active agent from 0.001 mg/kg to 10 mg/kg, typically around 0.1 mg/kg to 1 mg/kg, preferably about 1 mg/kg. A physician, in any event, will determine an actual dosage that is most suitable for an individual and will vary with the age, weight and response of the particular individual. The above dosages are exemplary of the average case. There can, of course, be individual instances where higher or lower dosage ranges are merited, and such are within the scope of this invention. It is preferred that the dosage is selected to modulate metabolism of the bacteria in such a way as to inhibit or stop growth of said bacteria or by killing said bacteria. The skilled artisan may identify this amount as provided herein as well as using other methods known in the art, e.g. by the application MIC tests.

A further embodiment of the invention provides for the contacting step of the methods to further comprise contacting an in-dwelling device in a patient. In-dwelling devices include, but are not limited to, surgical implants, prosthetic devices and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, and continuous ambulatory peritoneal dialysis (CAPD) catheters.

A quinolone, particularly a gemifloxacin compound or composition of the invention may be administered by injection to achieve a systemic effect against relevant bacteria, preferably a pathogenic Helicobacter bacteria, shortly before insertion of an in-dwelling device. Treatment may be continued after surgery during the in-body time of the device. In addition, the composition could also be used to broaden perioperative cover for any surgical technique to prevent bacterial wound infections caused by or related to pathogenic Helicobacter bacteria.

In addition to the therapy described above, a gemifloxacin compound or composition used in the methods of this invention may be used generally as a wound treatment agent to prevent adhesion of bacteria to matrix proteins, particularly pathogenic Helicobacter bacteria, exposed in wound tissue and for prophylactic use in dental treatment as an alternative to, or in conjunction with, antibiotic prophylaxis.

Alternatively, a quinolone, particularly a gemifloxacin compound or composition of the invention may be used to bathe an indwelling device immediately before insertion. The active agent will preferably be present at a concentration of 1 μg/ml to 10 mg/ml for bathing of wounds or indwelling devices. Also provided by the invention is a method of treating or preventing a bacterial infection by pathogenic Helicobacter bacteria comprising the step of administering an antibacterially effective amount of a composition comprising a quinolone, particularly a gemifloxacin compound to a mammal, preferably a human, suspected of having or being at risk of having an infection with pathogenic Helicobacter bacteria

While a preferred object of the invention provides a method wherein said pathogenic Helicobacter bacteria is selected from the group consisting of: Helicobacter sp., H. pylori, and pathogenic species of Helicobacter. Other pathogenic Helicobacter bacteria may also be included in the methods. The skilled artisan may identify these organisms as provided herein as well as using other methods known in the art, e.g. MIC tests.

For administration, the preferred composition of the invention include, among other things, methods wherein said composition comprises gemifloxacin, or a pharmaceutically acceptable derivative thereof. The invention also provides methods wherein the gemifloxacin compound is gemifloxacin or a pharmaceutically acceptable salt thereof, or a gemifloxacin compound such as gemifloxacin mesylate or a hydrate thereof, and/or the gemifloxacin compound is gemifloxacin mesylate sesquihydrate.

Each reference cited herein is hereby incorporated by reference in its entirety. Moreover, each patent application to which this application claims priority is hereby incorporated by reference in its entirety.

What is claimed is:

1. A method for modulating metabolism of pathogenic Helicobacter bacteria comprising the step of contacting pathogenic Helicobacter bacteria with an antibacterially effective amount of a composition comprising a gemifloxacin compound, or antibacterially effective derivatives thereof.

2. The method of claim 1 wherein said pathogenic Helicobacter bacteria is selected from the group consisting of: members of the genus Helicobacter, Helicobacter sp, H. pylori, and a pathogenic species of Helicobacter.

3. A method of treating or preventing a bacterial infection by pathogenic Helicobacter bacteria comprising the step of administering an antibacterially effective amount of a composition comprising a gemifloxacin compound to a mammal suspected of having or being at risk of having an infection with pathogenic Helicobacter bacteria.

4. The method of claim 3 wherein said pathogenic Helicobacter bacteria is selected from the group consisting of: members of the genus Helicobacter, Helicobacter sp, H. pylori, and a pathogenic species of Helicobacter.

5. The method of claim 1 wherein said modulating metabolism is inhibiting growth of said bacteria.

6. The method of claim 1 wherein said modulating metabolism is killing said bacteria.

7. The method of claim 1 wherein said contacting said bacteria comprises the further step of introducing said composition into a mammal.

8. The method of claim 3 wherein said mammal is a human.

9. The method of claim 7 wherein said mammal is a human.

10. The method of claim 8 wherein said bacteria is selected from the group consisting of: members of the genus Helicobacter, Helicobacter sp, H. pylori, and a pathogenic species of Helicobacter.

11. The method of claim 9 wherein said bacteria is selected from the group consisting of: members of the genus Helicobacter, Helicobacter sp, H. pylori, and a pathogenic species of Helicobacter.

12. The method according to claim 1 or 3 wherein the gemifloxacin compound is gemifloxacin or a pharmaceutically acceptable salt thereof.

13. The method according to claim 12 wherein the gemifloxacin compound is gemifloxacin mesylate or a hydrate thereof.

14. The method according to claim 13 wherein the gemifloxacin compound is gemifloxacin mesylate sesquihydrate.
